(19) United States
(12) Patent Application Publication
(10) Pub. No.: US 2020/0029791 A1
(43) Pub. Date: Jan. 30, 2020

(54) ENDOSCOPE

(71) Applicant: OLYMPUS CORPORATION, Tokyo (JP)

(72) Inventor: Nau SATAKE, Yokohama-shi (JP)

(73) Assignee: OLYMPUS CORPORATION, Tokyo (JP)

(21) Appl. No.: 16/535,177

(22) Filed: Aug. 8, 2019

Related U.S. Application Data
(63) Continuation of application No. PCT/JP2018/002356, filed on Jan. 25, 2018.

(30) Foreign Application Priority Data
Feb. 23, 2017 (JP) 2017-032271

(51) Int. Cl. A61B 1/00; A61B 1/045; A61B 1/05

(52) U.S. Cl. CPC A61B 1/00009; A61B 1/045; A61B 1/00096; A61B 1/00126; A61B 1/0011; A61B 1/00114; A61B 1/00045; A61B 1/05

(57) ABSTRACT

An endoscope includes an imaging device disposed on a distal end of an inserting portion of the endoscope, the imaging device including: an imaging element; a mounting board electrically connected to the imaging element; an assembly cable that includes a plurality of cables which are electrically connected to cable connection electrodes of the mounting board, and that includes a comprehensive coating configured to collectively coat the plurality of cables; and a heat shrinkable tube configured to coat the mounting board, connection portions to the plurality of cables, and outer circumference sides of the plurality of cables exposed by removal of the comprehensive coating, and an end portion on a proximal end side of the heat shrinkable tube having an inner circumference in contact with outer circumferences of the plurality of cables, and having an outer circumference in contact with an inner circumference of the comprehensive coating.

### CROSS-REFERENCE TO RELATED APPLICATION

This application is a continuation of International Application No. PCT/JP2018/002356, filed on Jan. 25, 2018, the entire contents of which are incorporated herein by reference.

### BACKGROUND

The present disclosure relates to an endoscope.

Heretofore, an endoscope that is inserted into a subject and performs observation for a subject region has been known, and has been widely used in a medical field or the like. In the endoscope, an assembly cable formed by bundling a plurality of cables is used, and is connected to a circuit board in a state in which an outer coating is removed therefrom.

In order to prevent an infectious disease or the like, the endoscope is subjected to disinfection and sterilization before being reused. In recent years, autoclave sterilization (high-pressure steam sterilization) has been adopted as a simple and inexpensive disinfection and sterilization method. The autoclave sterilization may give an influence to electronic components and the like. There is known a technique for sealing peripheries of the electronic components, cables and the like, which are mounted on a mounting board, with a sealing resin and then coating the peripheries with a heat shrinkable tube in order to reduce the influence.

### SUMMARY

An endoscope according to one aspect of the disclosure includes an imaging device disposed on a distal end of an inserting portion of the endoscope, the imaging device including: an imaging element configured to generate an electrical signal by receiving light formed as an image by an optical lens and performing photoelectric conversion for the received light; a mounting board disposed behind the imaging element on an optical axis of the optical lens and electrically connected to the imaging element; an assembly cable that includes a plurality of cables which individually transfer output signals from the imaging element or drive signals of the imaging element and are electrically connected to cable connection electrodes of the mounting board, and that includes a comprehensive coating configured to collectively coat the plurality of cables; and a heat shrinkable tube configured to coat the mounting board, connection portions to the plurality of cables, and outer circumference sides of the plurality of cables exposed by removal of the comprehensive coating, and an end portion on a proximal end side of the heat shrinkable tube having an inner circumference in contact with outer circumferences of the plurality of cables, and having an outer circumference in contact with an inner circumference of the comprehensive coating.

A method of manufacturing the endoscope according to one aspect of the disclosure includes: taking out a plurality of cables by removing a comprehensive coating on an end portion of an assembly cable, thereafter exposing core wires by removing sheaths of the plurality of cables, and connecting the core wires to a mounting board; fitting a shield frame to a holding frame of an optical lens, filling an inside of the shield frame and peripheries of the plurality of exposed cables with a sealing resin, and curing the sealing resin; shifting the comprehensive coating of the assembly cable, and removing an exposed binding tape and a shield layer; coating the shield frame and the sealing resin with a heat shrinkable tube, and thereafter shrinking the heat shrinkable tube; and shifting the comprehensive coating to cover an end portion of the shrunk heat shrinkable tube.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a view schematically illustrating an entire configuration of an endoscope system according to an embodiment;

FIG. 2 is a cross-sectional view of an imaging device for use in the endoscope of FIG. 1;

FIG. 3 is a cross-sectional view of the imaging device of FIG. 2, taken along a line A-A;

FIG. 4 is a cross-sectional view of the imaging device of FIG. 2, taken along a line B-B;

FIG. 5 is a view explaining a structure of the assembly cable;

FIG. 6A is a view explaining a manufacturing process of the imaging device according to the embodiment;

FIG. 6B is a view explaining the manufacturing process of the imaging device according to the embodiment;

FIG. 6C is a view explaining the manufacturing process of the imaging device according to the embodiment;

FIG. 6D is a view explaining the manufacturing process of the imaging device according to the embodiment;

FIG. 6E is a view explaining the manufacturing process of the imaging device according to the embodiment;

FIG. 6F is a view explaining the manufacturing process of the imaging device according to the embodiment;

FIG. 7 is a cross-sectional view of an imaging device according to a first modified example of the embodiment; and

FIG. 8 is a cross-sectional view of an imaging device according to a second modified example of the embodiment.

### DETAILED DESCRIPTION

An embodiment will be described below.

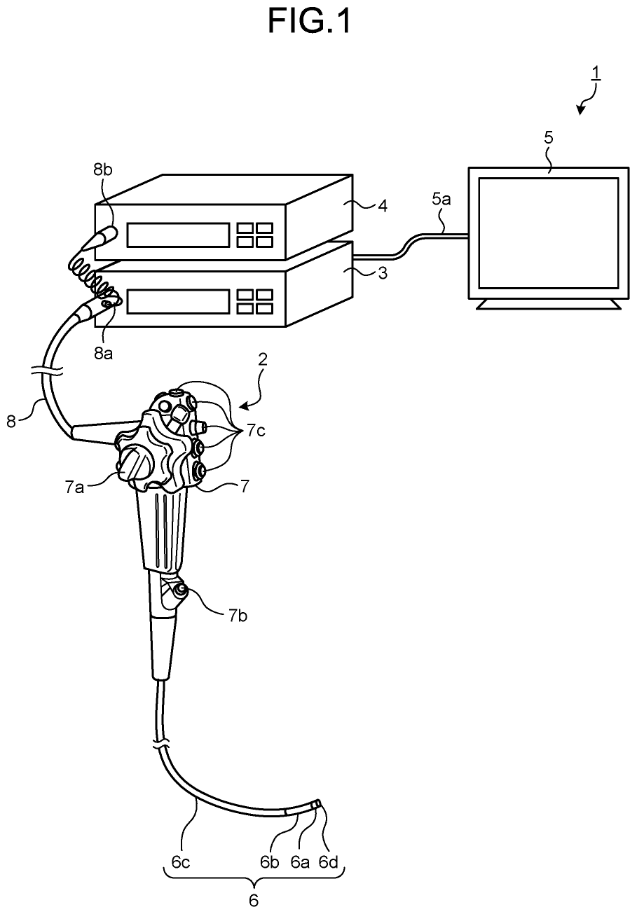

FIG. 1 is a view schematically illustrating an entire configuration of an endoscope system 1 according to an embodiment. As illustrated in FIG. 1, the endoscope system 1 according to the embodiment includes: an endoscope 2 that is introduced into a subject, captures an inside of the subject, and generates an image signal of the inside of the subject; an information processing device 3 (external processor) that implements predetermined image processing for such a signal of the image captured by the endoscope 2 and controls respective units of the endoscope system 1; a light source device 4 that generates illumination light of the endoscope 2; and a display device 5 that displays, as an image, the image signal already subjected to the image processing by the information processing device.

The endoscope 2 includes: an inserting portion 6 to be inserted into the subject; an operating unit 7 that is on a proximal end portion side of the inserting portion 6 and is grasped by an operator; and a flexible universal cord 8 that extends from the operating unit 7.

The inserting portion 6 is achieved by using an illumination fiber (light guide cable), an electric cable, an optical fiber, and the like. The inserting portion 6 has: a distal end portion 6a that incorporates an imaging unit to be described later therein; a curved portion 6b curvable and composed of a plurality of curved pieces; and a flexible tube portion 6c flexible and provided on a proximal end portion side of the curved portion 6b. The distal end portion 6a is provided with: an illumination portion that illuminates the inside of the subject through an illumination lens; an observation portion that captures the inside of the subject; an opening portion that allows communication of a treatment tool channel; and an air/water nozzle (not illustrated).

The operating unit 7 has: a curving knob 7a that curves the curved portion 6b in vertical and horizontal directions; a treatment tool inserting portion 7b from which treatment tools such as biological forceps and a laser scalpel are inserted into the body cavity of the subject; and a plurality of switch portions 7c that operates peripheral instruments such as the information processing device 3, the light source device 4, an air supply device, a water supply device and a gas supply device. The treatment tools inserted from the treatment tool inserting portion 7b appear from an opening portion 6d, which is located at the distal end of the inserting portion 6, through the treatment tool channel provided in an inside.

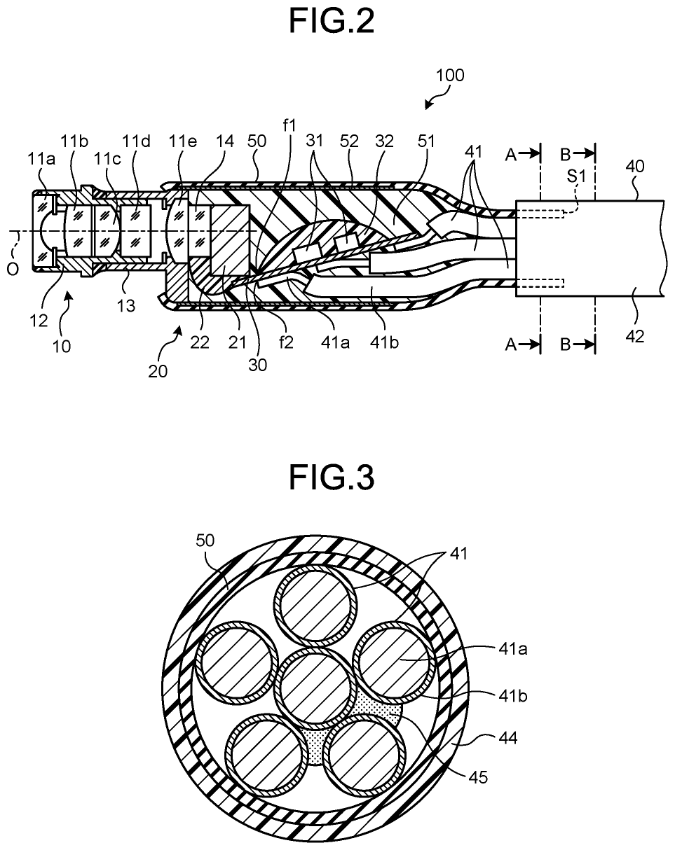

The universal cord 8 is composed by using the illumination fiber, the cable and the like. The universal cord 8 branches at a proximal end thereof, an end portion of one of the branched proximal end is a connector 8a, and an end portion of the other thereof is a connector 8b. The connector 8a is detachable from a connector of the information processing device 3. The connector 8b is detachable from the light source device 4. The universal cord 8 propagates illumination light, which is emitted from the light source device 4, to the distal end portion 6a via the connector 8b and the illumination fiber. Moreover, the universal cord 8 transmits an image signal, which is captured by an imaging device 100 (refer to FIG. 2) to be described later, to the information processing device 3 via the cable and the connector 8a.

The information processing device 3 implements predetermined image processing for the image signal output from the connector 8a, and controls the entire endoscope system 1.

The light source device 4 is composed by using a light source that emits light, a condenser lens and the like. Under control of the information processing device 3, the light source device 4 emits light from the light source, and supplies the endoscope 2, which is connected thereto via the connector 8b and the illumination fiber of the universal cord 8, with the light as illumination light to the inside of the subject as an object to be captured.

The display device 5 is composed by using a displaying display using liquid crystal or organic electroluminescence (EL). Via a video cable 5a, the display device 5 displays various pieces of information including the image subjected to the predetermined image processing by the information processing device 3. Thus, the operator operates the endoscope 2 while seeing the image (in-vivo image) displayed by the display device 5, and can thereby observe a desired position in the subject, and can determine a condition there.

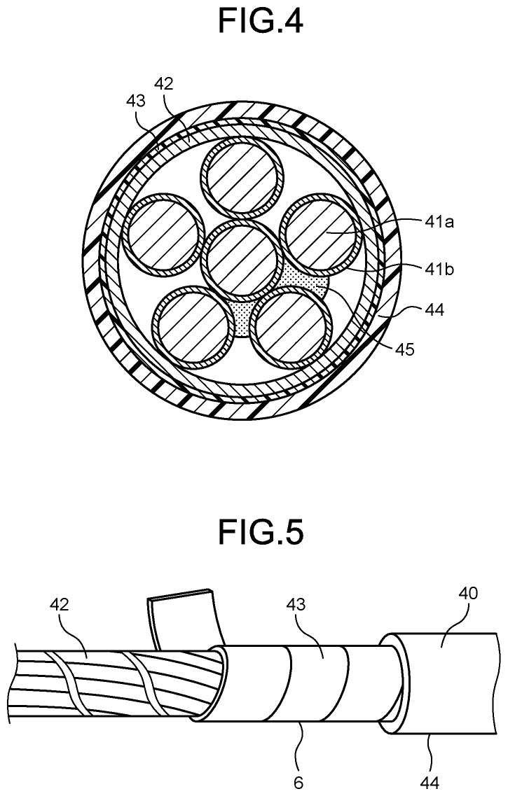

Next, a configuration of the imaging device 100 will be described in detail. FIG. 2 is a cross-sectional view of the imaging device 100 for use in the endoscope 2 of FIG. 1. FIG. 3 is a cross-sectional view of the imaging device 100 of FIG. 2, taken along a line A-A. FIG. 4 is a cross-sectional view of the imaging device 100 of FIG. 2, taken along a line B-B. FIG. 5 is a view explaining a structure of an assembly cable 40. Note that, though FIG. 2 is a cross-sectional view, cables 41 and the assembly cable 40 are illustrated not by the cross-sectional view but a side view.

The imaging device 100 is composed of: a lens unit 10 having a plurality of optical lenses 11a to 11e; and an imaging unit 20 having an imaging element 21. Herein, the lens unit 10 has an optical axis O.

The lens unit 10 has: the plurality of optical lenses 11a to 11e; a first lens holding frame 12 that holds the optical lenses 11a to 11d; and a second lens holding frame 13 that holds the optical lens 11e. The first lens holding frame 12 is fitted into the second lens holding frame 13. Light that has transmitted through the plurality of optical lenses 11a to 11e of the lens unit 10 is condensed on a light receiving surface of the imaging element 21, and forms an optical image.

The imaging unit 20 includes an imaging element 21, a mounting board 30 formed of a flexible printed board, and the assembly cable 40.

The imaging element 21 is adhered and fixed to the optical lens 11e via a filter 14. The mounting board 30 is disposed behind the imaging element 21 on the optical axis O, and has an inner lead (not illustrated) electrically connected to an electrode pad (not illustrated) of the imaging element 21. A periphery of such a connection portion is protected by an adhesive 22.

Electronic components 31 are mounted on an upper surface f1 of the mounting board 30, and a plurality of the cables 41 are connected to a lower surface f2. A periphery of the electronic components 31 mounted on the mounting board 30 is sealed by an adhesive 32.

As illustrated in FIG. 4 and FIG. 5, the assembly cable 40 has: the cables 41 as simple wires in which core wires 41a as conductor portions are coated with insulating sheaths 41b; a shield layer 42 that is disposed around the plurality of cables 41 and electrically shields the cables 41; a binding tape 43 that binds the shield layer 42; and a comprehensive coating 44 that coats the binding tape 43. In the assembly cable 40, the comprehensive coating 44 is removed at an end portion thereof, and the cables 41 are drawn therefrom. In the cables 41, the core wires 41a exposed by removal of the sheaths 41b at end portions thereof are connected to the lower surface f2 of the mounting board 30 via solder (not illustrated) or the like.

To an outer circumferential portion of the second lens holding frame 13, a metal-made shield frame 52 that electrically shields the imaging element 21 and the electronic components 31 is fitted. Moreover, an inside of the shield frame 52 and peripheries of the cables 41 are sealed with a sealing resin 51.

A heat shrinkable tube 50 is disposed around the shield frame 52. In the heat shrinkable tube 50, a front end thereof is located on the second lens holding frame 13, and a rear end thereof is located at a position S1 in the comprehensive coating 44 of the assembly cable 40. In this embodiment, the shield layer 42 and the binding tape 43 on the end portion of the assembly cable 40 are partially cut off, and the heat shrinkable tube 50 is inserted into a gap thus formed. Hence, on the cross section taken along the line A-A illustrated in FIG. 3, the heat shrinkable tube 50 is present on outer circumferences of the cables 41, and the comprehensive coating 44 is present on an outer circumference of the heat shrinkable tube 50.

In this embodiment, the shield layer 42 and the binding tape 43 on the end portion of the assembly cable 40 are partially cut off, and the heat shrinkable tube 50 is inserted into the formed gap, whereby an outer circumference of the assembly cable 40 can be prevented from thickening. Note that, in a case of using the general assembly cable 40, for example, in which a binding diameter of the cables 41 is 0.9 mm, a thickness of the shield layer 42 is 0.05 mm, a thickness of the binding tape 43 is 0.05 mm, a thickness of the comprehensive coating 44 is 0.1 mm, and a diameter of the assembly cable 40 is 1.3 mm, and using the heat shrinkable tube 50 in which a thickness before shrinkage is 0.01 mm, and a thickness after the shrinkage is approximately 0.02 mm, it is relatively easily possible to cut off the shield layer 42 and the binding tape 43, and to insert the heat shrinkable tube 50 into the formed gap. Depending on cases, the heat shrinkable tube 50 may be inserted into a gap formed by cutting off only the binding tape 43 without cutting off the shield layer 42. Alternatively, while an interposition 45 is disposed in the assembly cable 40 for the purpose of fixing the disposition of the respective cables 41, and so on, this interposition 45 may be removed together with the shield layer 42 and the binding tape 43, and a gap into which the heat shrinkable tube 50 is inserted may be formed.

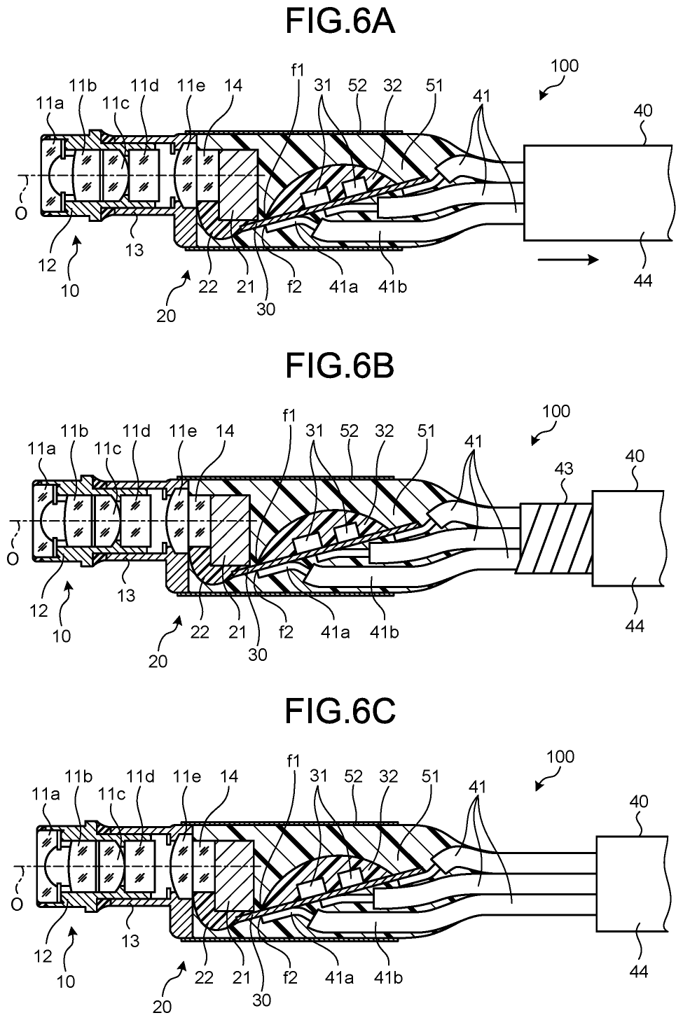

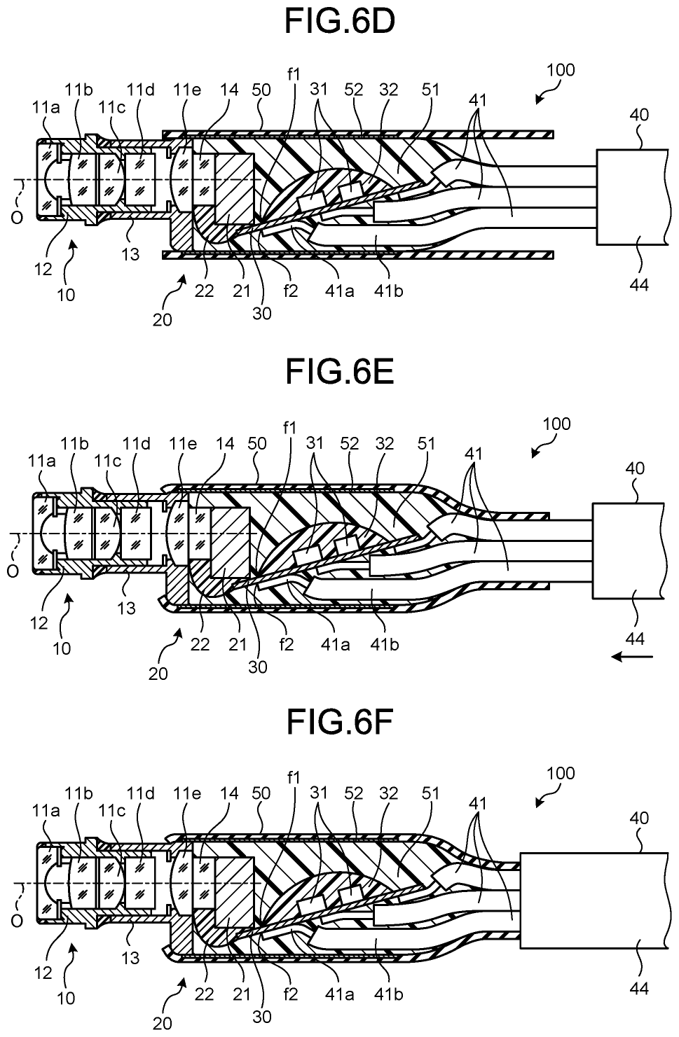

Next, a method of manufacturing the imaging device 100 according to this embodiment will be described with reference to the drawings. FIG. 6A to FIG. 6F are views explaining a manufacturing process of the imaging device 100 according to the embodiment. Note that, though FIG. 6A to FIG. 6F are cross-sectional views, the cables 41 and the assembly cable 40 are illustrated not by the cross-sectional view but side views like FIG. 2.

First, the comprehensive coating 44 on the end portion of the assembly cable 40 is removed to take out the cables 41, the sheaths 41b of the cables 41 are removed to expose the core wires 41a, and the core wires 41a are connected to the mounting board 30. After the connection, the shield frame 52 is fitted to the second lens holding frame 13, and the peripheries of the shield frame 52 and the exposed cables 41 are filled with the sealing resin 51, which is then cured. After the sealing resin 51 is cured, the comprehensive coating 44 of the assembly cable 40 is shifted to a proximal end side illustrated in an arrow of FIG. 6A, and the binding tape 43 is exposed as illustrated in FIG. 6B. The exposed binding tape 43 is removed, and the shield layer 42 exposed by the removal of the binding tape 43 is removed similarly (see FIG. 6C).

Thereafter, as illustrated in FIG. 6D, the shield frame 52 is coated with the heat shrinkable tube 50, which is then shrunk. After the heat shrinkable tube 50 is shrunk, the comprehensive coating 44 is shifted to a distal end side illustrated by an arrow of FIG. 6E, and the heat shrinkable tube 50 is inserted into a gap formed by removing the binding tape 43 and the shield layer 42 (see FIG. 6F).

When a thickness of the heat shrinkable tube 50 after the shrinkage is equal to or less than a sum of the thicknesses of the binding tape 43 and the shield layer 42, the heat shrinkable tube 50 is inserted into the gap formed by removing the binding tape 43 and the shield layer 42, whereby a diameter of the circumference of the assembly cable 40 can be prevented from increasing. In the above, the binding tape 43 and the shield layer 42 in the assembly cable 40 are removed after the peripheries of the shield frame 52 and the exposed cables 41 are sealed with the sealing resin 51; however, after the binding tape 43 and the shield layer 42 are removed, the cables 41 may be connected to the mounting board 30, the shield frame 52 may be fitted to the second lens holding frame 13, and the peripheries of the shield frame 52 and the exposed cables 41 may be filled with the sealing resin 51.

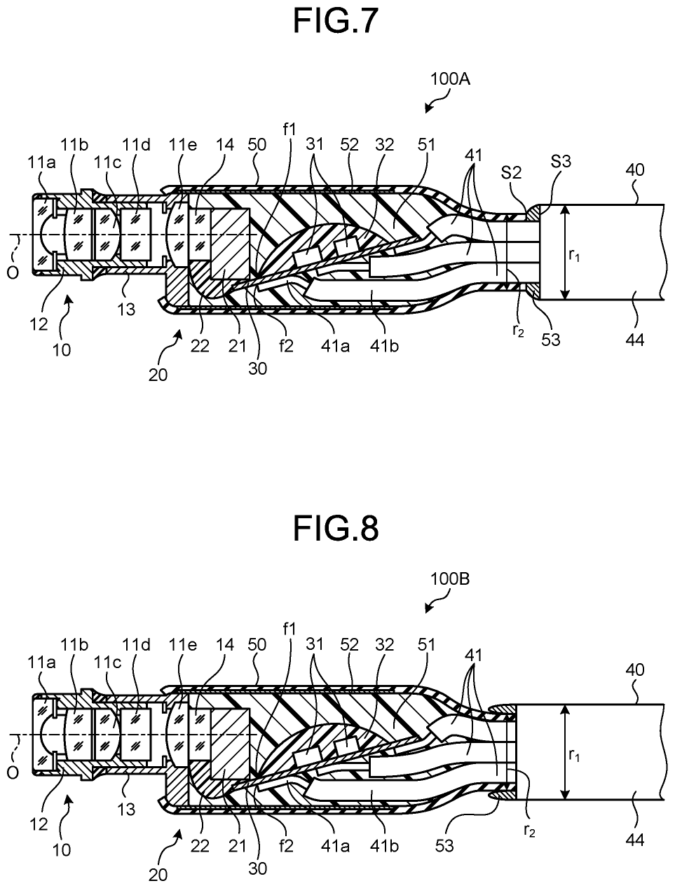

Note that, in a case of using a thicker heat shrinkable tube 50, the heat shrinkable tube 50 and the comprehensive coating 44 may be adhered and fixed to each other by an adhesive without inserting the heat shrinkable tube 50 into the comprehensive coating 44 of the assembly cable 40. FIG. 7 is a cross-sectional view of an imaging device 100A according to a first modified example of this embodiment. Note that, though FIG. 7 is a cross-sectional view, the cables 41 and the assembly cable 40 are illustrated not by the cross-sectional view but a side view like FIG. 2.

Like the embodiment, in the first modified example, the heat shrinkable tube 50 directly coats outer circumference sides of the cables 41 exposed by removal of the comprehensive coating 44; however, an end portion S2 on a proximal end side of the heat shrinkable tube 50 is located closer to the imaging element 21 than an end portion S3 on a distal end side of the comprehensive coating 44. Note that, herein, unless the sealing resin 51 leaks out from the heat shrinkable tube 50, the sealing resin 51 may enter between the heat shrinkable tube 50 and the cables 41 exposed by the removal of the comprehensive coating 44. A space is present between the end portion S2 on the proximal end side of the heat shrinkable tube 50 and the end portion S3 on the distal end side of the comprehensive coating 44, and the space is filled with an adhesive 53, which adheres and fixes the heat shrinkable tube 50 and the comprehensive coating 44 to each other.

Like the first embodiment, in the first modified example, the diameter of the circumference of the assembly cable 40 can be prevented from increasing. Note that, if an outer diameter r2 on the proximal end side of the heat shrinkable tube 50 is equal to or less than an outer diameter r1 of the assembly cable 40 in the first modified example, then a circumference of the proximal end side of the heat shrinkable tube 50 is thinned, and the circumference of the proximal end side of the heat shrinkable tube 50 also becomes usable as a disposing space of a light guide and the like.

Moreover, the end portion on the proximal end side of the heat shrinkable tube 50 may be disposed so as to be brought into contact with the end portion on the distal end side of the comprehensive coating 44. FIG. 8 is a cross-sectional view of an imaging device 100B according to a second modified example of this embodiment. Note that, though FIG. 8 is a cross-sectional view, the cables 41 and the assembly cable 40 are illustrated not by the cross-sectional view but a side view like FIG. 2.

In the second modified example, the heat shrinkable tube 50 directly coats the outer circumference sides of the cables 41 exposed by the removal of the comprehensive coating 44, and the end portion on the proximal end side of the heat shrinkable tube 50 is in contact with the end portion on the distal end side of the comprehensive coating 44. The outer diameter r2 on the proximal end side of the heat shrinkable tube 50 is smaller than the outer diameter r1 of the assembly cable 40, and the heat shrinkable tube 50 and the comprehensive coating 44 are adhered and fixed to each other by the adhesive 53 disposed on the outer circumference on the proximal end side of the heat shrinkable tube 50.

Note that, also in FIG. 2 and FIG. 6F, though not illustrated, the heat shrinkable tube 50 and the comprehensive coating 44 are adhered and fixed to each other similarly by the adhesive 53 disposed on the outer circumference on the proximal end side of the heat shrinkable tube 50.

Like the first embodiment and the first modified example, in the second modified example, the diameter of the circumference of the assembly cable 40 can be prevented from increasing.

The case of using the single heat shrinkable tube 50 has been described above; however, also in a case of using two heat shrinkable tubes on the distal end side and the rear end side, a similar effect can be obtained by directly coating, with the heat shrinkable tube on the rear end side, the outer circumference sides of the cables 41 exposed by the removal of the comprehensive coating 44.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope comprising an imaging device disposed on a distal end of an inserting portion of the endoscope,
the imaging device including:
an imaging element configured to generate an electrical signal by receiving light formed as an image by an optical lens and performing photoelectric conversion for the received light;
a mounting board disposed behind the imaging element on an optical axis of the optical lens and electrically connected to the imaging element;
an assembly cable that includes a plurality of cables which individually transfer output signals from the imaging element or drive signals of the imaging element and are electrically connected to cable connection electrodes of the mounting board, and that includes a comprehensive coating configured to collectively coat the plurality of cables; and
a heat shrinkable tube configured to coat the mounting board, connection portions to the plurality of cables, and outer circumference sides of the plurality of cables exposed by removal of the comprehensive coating, and
an end portion on a proximal end side of the heat shrinkable tube having an inner circumference in contact with outer circumferences of the plurality of cables, and having an outer circumference in contact with an inner circumference of the comprehensive coating.

2. The endoscope according to claim 1, wherein the assembly cable includes a shield layer configured to electrically shield the plurality of cables, a binding tape configured to bind the shield layer, and the comprehensive coating configured to coat the binding tape.

3. A method of manufacturing the endoscope according to claim 1, comprising:
taking out a plurality of cables by removing a comprehensive coating on an end portion of an assembly cable, thereafter exposing core wires by removing sheaths of the plurality of cables, and connecting the core wires to a mounting board;
fitting a shield frame to a holding frame of an optical lens, filling an inside of the shield frame and peripheries of the plurality of exposed cables with a sealing resin, and curing the sealing resin;
shifting the comprehensive coating of the assembly cable, and removing an exposed binding tape and a shield layer;
coating the shield frame and the sealing resin with a heat shrinkable tube, and thereafter shrinking the heat shrinkable tube; and
shifting the comprehensive coating to cover an end portion of the shrunk heat shrinkable tube.
